(19) United States
(12) Patent Application Publication
(10) Pub. No.: US 2018/0298244 A1
(43) Pub. Date: Oct. 18, 2018

(54) ADHESIVE COMPOSITION CONTAINING A STARCH HYDROLYSATE FOR HEAT-SEALING

(71) Applicant: TEREOS STARCH & SWEETENERS BELGIUM, Aalst (BE)

(72) Inventor: Andreas REDL, Aalst (BE)

(21) Appl. No.: 15/767,103

(22) PCT Filed: Oct. 7, 2016
(86) PCT No.: PCT/IB2016/056015

§ 371 Date: Apr. 9, 2018

(30) Foreign Application Priority Data
Oct. 7, 2015 (BE) BE-2015/5636

(51) Int. Cl. C09J 103/02; A61F 13/15; A61L 15/28; A61L 15/58; C09J 123/06; B32B 5/02; B32B 9/04; B32B 5/26; B32B 9/02; D04H 11/00

(52) U.S. Cl. CPC C09J 103/02; A61F 13/15739; A61L 15/28; A61L 15/58; C09J 123/06; B32B 5/022; D10B 2201/02; B32B 5/26; B32B 9/02; D04H 11/00; B32B 2262/062; B32B 2555/00; B32B 9/047

(57) ABSTRACT

A method for thermal bonding of a fibrous matrix to a support, comprising the steps of: i) deposition, on a bonding region of said support and/or of said fibrous matrix, of a pulverulent composition comprising essentially a starch hydrolysate having a DE of greater than 30, ii) bringing the bonding regions of said fibrous matrix and of said support into contact, iii) applying, to one and/or the other of the bonding regions of said support and of said fibrous matrix, a temperature of between 100 and 180° C. in order to bond said fibrous matrix to the support. The use of an adhesive composition comprising essentially a starch hydrolysate having a DE of greater than 30 in thermal bonding and an adhesive composition for binding a fibrous matrix to a support.

This application claims the benefit of Belgian patent application No. BE-2015/5636, filed Oct. 7, 2015, which is hereby incorporated by reference in its entirety.

### TECHNICAL FIELD

The present disclosure relates to a method for bonding a fibrous matrix to a support by applying a starch hydrolysate having a DE of greater than 30 to a support, the use of an adhesive composition comprising essentially a starch hydrolysate having a DE of greater than 30 in thermal bonding, and an adhesive composition comprising more than 60% of a starch hydrolysate having a DE of greater than 30 for attaching a fibrous matrix to a support.

### DETAILED DESCRIPTION OF THE INVENTION

Hygiene products generally consist of a series of sheets having protective or absorbent functions. These different sheets are linked to one another by thermal bonding.

Generally, thermoplastic polyethylene is widely used in the bonding of cellulose matrices. The thermoplastic bonding of cellulose matrices requires the uniform application of small amounts of adhesive.

An aim of embodiments of the present invention is to replace the petroleum derivatives largely used in thermal bonding with bio-based products with a low level of allergenicity, enabling use in disposable hygiene products, especially intended for contact with the skin, especially repeated and/or prolonged contact with the skin. The introduction of plastic materials and especially materials containing microplastics in disposable hygiene products is an increasingly worrying matter, in terms of both protecting public health and protecting the environment. Thus, within the context of disposable hygiene products, while the cellulose matrices thereof are biodegradable, the introduction of plastic materials and especially of microplastics within these matrices during the assembly thereof makes them potentially environmentally damaging products. Indeed, in order to obtain a suitable adhesive, the thermoplastic products used are finely ground, forming microplastics that are most likely to be spread into the environment without being broken down. In addition, these powders are difficult to obtain, due to the low melting point of these plastics, and are therefore relatively expensive.

The invention in embodiments relates to a method for thermal bonding of a fibrous matrix, preferentially a cellulose matrix, to a support, comprising the steps of:
deposition, on a bonding region of said support and/or of said fibrous matrix, of a pulverulent composition comprising essentially a starch hydrolysate having a dextrose equivalent (DE) of greater than 30, preferentially of between 30 and 90, more preferentially 36 to 40;
bringing the bonding regions of said fibrous matrix and of said support into contact;
applying, to one and/or the other of the bonding regions of said support and of said fibrous matrix, a temperature of between 100 and 180° C. in order to bond said fibrous matrix to the support; preferentially, the step of bringing the bonding regions into contact and the step of applying temperature to one and/or the other of the bonding regions may be simultaneous or successive.

This method is particularly advantageous in the manufacture of hygiene products and especially disposable absorbent articles, especially because said pulverulent composition comprising essentially a starch hydrolysate having a DE of greater than 30 is essentially of natural biodegradable origin. “Essentially of natural biodegradable origin” is intended to mean a composition comprising less than 20% of petroleum-based products such as synthetic polymers or resins and/or of chemical additives (bridging agents, pigments, etc.), typically less than 15%, 10%, 5%, 2% or 1% (w/w) of petroleum-based products and/or of chemical additives.

“Starch hydrolysate” is intended to mean a carbohydrate composition which comprises dextrose, maltose, maltotrioses and polysaccharides. Standard starch hydrolysates are produced by acid or enzymatic hydrolysis of starch, regardless of its botanical origin (for example cereals, leguminous plants or tuberous plants). In fact, they are a mixture of glucose and of glucose polymers, with extremely varied molecular weights. Starch hydrolysates are generally classified as a function of their reducing power, expressed by the concept of dextrose equivalent or DE.

As it is used here, the expression “dextrose equivalent” or “DE” refers to the “content of reducing sugars, expressed as dextrose percentage on dry matter” and is used to characterize the molecular weight of the polysaccharides. The theoretical value thereof is inversely proportional to the average molecular weight (Mn). It is calculated as follows:

DE=Mglucose/Mn×100 or

DE=180/Mn×100

Thus, dextrose has a DE of 100 while pure starch (for example corn starch) has a value of 0.

Typically, the starch hydrolysate according to the invention has a DE of between 31 and 85, more preferentially between 32 and 80; 33 and 75; 34 and 70; 35 and 60; 36 and 50; 36 and 45.

The expression “composition comprising essentially” refers to a composition comprising more than 50% (w/w) of a starch hydrolysate, preferentially more than 60% (w/w), more than 70%, 80%, 90%, 95%, 98% of a starch hydrolysate. Typically, the composition comprises from 51 to 100%, 55 to 98%, 60 to 97%, 70 to 95% of starch hydrolysate.

Typically, the adhesive composition according to the invention is an amorphous pulverulent composition. “Amorphous” is intended to mean a non-crystalline composition; for the purposes of the present invention, such an amorphous composition may be obtained by spray drying and/or granulation of a liquid and/or pulverulent starch hydrolysate. Such an amorphous composition may also be obtained by rapid cooling of a molten starch hydrolysate (what is referred to as the “chilled belt” process).

The pulverulent composition according to embodiments of the invention is advantageous in that its pulverulent form makes it possible to overcome problems associated with the viscosity or the preservation of the adhesive composition. Indeed, the advantage of such a composition is its stability; it does not have to be dissolved before use, nor to be stirred and kept hot. It may be finely metered and applied by simple deposition. The spraying of such a composition or the use in liquid form would not enable a uniform deposition of the adhesive on the matrix. In addition, in light of the viscosity of the mixture obtained, spraying would not enable droplets to be obtained that are sufficiently small to avoid any irregularity in the bonding. Indeed, the pulverulent form enables fine application of the amount of adhesive required for bonding. Finally, the powder form makes it possible to overcome the consequences associated with the potential hydrophobicity of the fibres of the fibrous matrix.

Preferentially, the pulverulent composition has a mean particle size distribution (D(v,0.5)) of between 40 and 500 μm, preferentially from 50 to 450 μm, more preferentially from 150 to 400 μm. Typically, the composition according to embodiments of the invention comprises more than 90% of particles having a particle size distribution of greater than 40 μm and/or more than 15% of particles having a particle size distribution of greater than 250 μm and/or less than 10% of particles having a particle size distribution of greater than 500 μm. Advantageously, the pulverulent composition is composed of agglomerated particles; typically, it is formed of particles obtained by wet granulation. The particle size distribution may be analysed by various techniques, such as Alpine and/or Retsch. The particle size distribution is preferentially measured by the Retsch method, according to the method described in the European Pharmacopoeia 6.0 No. 01/2008: 20938, p.325. More particularly, the particle size distribution of the powder according to the invention may be measured by means of a Retsch sieve shaker, model AS200, controlled ‘g’ according to the manufacturer's recommendations.

As used in the present document, the term “thermal bonding” refers to the attachment, welding or adhesion of a material to itself or to another by raising the temperature (beyond room temperature). For the purposes of the present invention, this expression refers to welding or to acquiring adhesion between the bonding region of the fibrous matrix and the bonding region of the support by applying a rise in temperature to one and/or the other of the bonding regions. Advantageously, the bonding temperature is between 100 and 180° C., preferentially between 105 and 170° C., even more preferentially between 110 and 160° C. Typically, the increase in the temperature may be combined with the application of pressure to one and/or the other of the bonding regions of the support and of the fibrous matrix. Advantageously, a pressure of 1 to 100 bar, typically 2 to 7 bar, advantageously 1 to 8 bar, is applied to one and/or the other of the bonding regions of the support and of the fibrous matrix simultaneously and/or subsequently to the application of temperature; preferentially, the pressure applied is between 2 and 60 bar, even more preferentially between 3 and 55 bar. The increase in the temperature and the application of pressure to at least one of the bonding regions may be carried out independently of one another, for distinct durations. Typically, the duration of the increase in temperature and of the application of pressure to one and/or the other of the bonding regions are, independently of one another, between 1 millisecond and 10 min, preferentially between 0.1 second and 1 minute, even more preferentially between 0.5 and 30 seconds; 1 and 20 seconds; 2 and 10 seconds. Advantageously, a pressure of between 1 and 100 bar, typically 2 to 70 bar, and a temperature of between 100 and 180° C., are applied concomitantly to one and/or the other of the bonding regions for at least 1 millisecond and 10 min, preferentially between 0.5 and 30 seconds, more preferentially between 1 and 20 seconds; 2 and 10 seconds.

For the purposes of the present invention, the expression “bonding regions” corresponds to the surface of said fibrous matrix or of said support intended to be thermally bonded.

“Support” is intended to mean a second fibrous matrix. Preferentially, the plastic material is selected from polyamide (PA), polyurethane (PUR), polypropylene (PP), poly(ethylene-co-vinyl alcohol) (EVOH), polyvinyl alcohol (PVOH), polyethylene terephthalate (PET), polyethylene naphthalate (PEN), polystyrene (PS), polymethyl methacrylate (PMMA), poly(vinyl chloride) (PVC), polyethylene (PE) and combinations thereof.

The expression “fibrous matrix” refers to natural fibres (for example wood or cotton fibres), synthetic fibres (for example, polyester or polypropylene fibres) or a combination of natural and/or synthetic fibres. Said fibrous matrix may be woven, nonwoven, spun, embossed, or knitted. The fibres used in the manufacture of matrices suitable for use in embodiments of the present invention are generally wood pulp fibres, viscose fibres or cotton fibres. However, other cellulose fibres, and also mixtures of cellulose fibres with fibres of a different (natural and/or synthetic) origin may be used. Thus, fibres suitable for use in embodiments of the present invention may be selected from cellulose fibres, silk, wool, polyester, polypropylene, polyethylene, polyamide (such as nylon), cellulose acetate, polyvinyl fluoride, polyvinylidene chloride, acrylics (such as Orlon), polyvinyl acetate, insoluble polyvinyl alcohol, analogues thereof and combinations thereof. By way of illustration, advantageous combinations of synthetic fibres are polypropylene/polyethylene, polyester/polyethylene, polyester/polypropylene, polyamide/polyester, polyamide/polyethylene, polyamid/polypropylene.

Preferentially, the fibrous matrix has a thickness of between approximately 0.012 mm and approximately 0.05 cm, preferentially a thickness of between approximately 0.02 mm and approximately 0.03 cm, even more preferentially of between 0.05 mm and 0.01 cm; between 0.1 mm and 5 mm; between 0.5 mm and 3 mm.

The invention in embodiments also relates to the use of said pulverulent composition according to embodiments of the invention for the thermal bonding of a fibrous matrix, preferentially a cellulose fibrous matrix, to a support.

The invention in embodiments also relates to a hygiene product able to be obtained by carrying out the method according to embodiments of the invention, said hygiene product comprising at least one fibrous matrix thermally bonded to a support by a pulverulent composition comprising essentially a starch hydrolysate having a DE of greater than 30.

“Hygiene product” is intended to mean any article used for the bodily hygiene of a human or an animal, especially disposable hygiene articles, especially disposable absorbent articles, typically selected from compresses, tampons, wipes, dressings, sanitary towels, mattress protectors, pantyliners, babies' nappies, articles for adult incontinence, and sweat pads.

The method according to embodiments of the invention is particularly advantageous in that it enables easy separation of the support and the fibrous matrix. The adhesion strength may be altered as a function of the adhesive added to the starch hydrolysate having a DE of greater than 30. Typically, the invention in embodiments finally relates to an adhesive composition comprising more than 60% (w/w) of a starch hydrolysate having a DE of greater than 30 and an adhesive selected from polyethylene, polyethylene, polypropylene, polyamide, polyvinyl alcohol and/or copolyester. Typically, the adhesive chosen has a melting point of between 100 and 180° C. Advantageously, the composition comprises 0.1 to 40% of adhesive, preferentially 0.2 to 30%, 0.5 to 20%, 0.7 to 15%, 1 to 10%, 1.2 to 7%, 1.5 to 5% by weight of adhesive.

Although they have distinct meanings, the terms “comprising”, “containing”, “including” and “consisting of” have been used interchangeably in the description of the invention, and may be replaced by one another.

The invention will be better understood on reading the following examples, given solely by way of illustration.

### EXAMPLE

### Materials and Methods

The adhesive compositions tested are as follows:
pulverulent PE sold by Abifor
pulverulent maltodextrin, DE 19, sold by Tereos Syral under the trade name MALDEX® 190
pulverulent glucose obtained by spray drying and sold by Tereos Syral under the trade name MERITOSE® SD 250
pulverulent glucose syrup, DE 38, sold by Tereos Syral under the trade name GLUCODRY® G 380

On a thin nonwoven cotton sheet having a surface area of 10 cm², approximately 1 g of powder is distributed uniformly on a bonding surface. The sheet prepared in this way is then covered with a second nonwoven cotton sheet and placed between two sheets of paper. The sheet structure is then placed in a press heated to 150° C. (VOGT Labopress 200T Vogt Maschinenbau GmbH, Brunsbüttler barrage 114, D 13581 Berlin) for 5 seconds at an operating pressure of 50 bar.

After exiting the press, after a cooling time of approximately 5 minutes, the adhesion between the thin cotton sheets is tested by tearing the sheets apart until the adhesion region breaks.

Good adhesion is characterized by sheets which can only be separated by at least partial tearing of the bonding region of one of the sheets. Poor adhesion, or the lack of adhesion, is characterized by the very low, or absent, resistance to separation of the sheets.

### Results

| Adhesive composition | Adhesion |
|---|---|
| Negative control | — |
| Pulverulent PE | Good adhesion |
| Pulverulent maltodextrin, DE 19 | No adhesion |
| Pulverulent glucose | Weak adhesion |
| Pulverulent glucose syrup, DE 38 | Good adhesion |

In the case of the pulverulent glucose, which has a dextrose equivalent of 100, melting of the powder upon heating is indeed observed, but the composition obtained does not enable the adhesion of the two sheets, and migrates towards the more absorbent layers, in this instance the paper.

Moreover, it is observed that, compared to the pulverulent PE, the deposition of the dehydrated glucose syrup is more difficult due to the fine particle size distribution. The inventors have demonstrated that, compared to the non-agglomerated form, agglomerated pulverulent glucose syrups make it possible to improve the precision of the regions for deposition of the adhesive composition on the surface of the sheet.

The inventors have shown that the pulverulent formulation, enabling the best control of the distribution of the powder and also of the amounts deposited, had more than 90% of particles having a particle size distribution of greater than 40 μm, more than 15% of particles having a particle size distribution of greater than 250 μm and less than 10% of particles having a particle size distribution of greater than 500 μm.

Different heating temperatures were tested; a temperature between 100 and 150° C. enables better results to be observed with the pulverulent glucose syrup, DE 37.

1. A method for thermal bonding of a fibrous matrix to a support, comprising the steps of:
deposition, on a bonding region of said support and/or of said fibrous matrix, of a pulverulent composition comprising more than 50% (w/w) of a starch hydrolysate having a dextrose equivalent (DE) of between 30 and 90;
bringing the bonding regions of said fibrous matrix and of said support into contact;
applying, to one and/or the other of the bonding regions of said support and of said fibrous matrix, a temperature of between 100 and 180° C. in order to bond said fibrous matrix to the support.

2. The method according to claim 1, characterized in that said pulverulent composition has a DE of between 36 to 40.

3. The method according to claim 1, characterized in that said pulverulent composition comprises more than 60% (w/w) of starch hydrolysate.

4. The method according to claim 1, characterized in that said pulverant composition has a mean particle size distribution of between 40 and 500 pm.

5. The method according to claim 1, characterized in that a pressure of 1 to 100 bar is applied to at least one of the bonding regions of said support and/or of said fibrous matrix during the step of applying a temperature of between 100 and 180° C. to said bonding region(s).

6. The method according to claim 1, characterized in that said fibrous matrix has cellulose fibres.

7. A method of usin a pulverulent composition comprising more than 50% (w/w) of a starch hydrolysate having a dextrose equivalent (DE) of between 30 and 90, comprising thermally bonding a fibrous matrix to a support with the pulverant composition.

8. A hygiene product, comprising at least one fibrous matrix thermally bonded to a support by a pulverulent composition comprising more than 50% (w/w) of a starch hydrolysate having a dextrose equivalent (DE) of between 30 and 90.

9. The hygiene product according to claim 8, characterized in that said hygiene product is a disposable absorbent article, preferentially selected from compresses, tampons, wipes, dressings, sanitary towels, mattress protectors, pantyliners, babies' nappies, articles for adult incontinence, and sweat pads.

10. A pulverulent adhesive composition comprising more than 50% (w/w) of a starch hydrolysate having a dextrose equivalent (DE) of between 30 and 90 and an adhesive selected from polyethylene, polypropylene, polyamide, polyvinyl alcohol and/or copolyester.
